SEQUENCE LISTING (24 sequences)

SEQ ID NO: 1 - DNA, 501 bp, Homo sapiens
gggcaacagagaaagattctatctcaaaaaaaaaaatttttttttaagttaaaaataaaataaagactttggggcaatacagggggtcctgggagtgtaacccataacccccaagagtgatttctgcaatctcgtttcaaattacagggtcaactgctatgatgtgtttggagcccagtcaccctttggtggctacaagatgtcggggagtggccgggagttgggcgagtacgggctgcaggcatacactgaagtgaaaactgtgagtgtgggacctgctgggggctcagggcctgttggggcttgagggtctgctggtggctcggagcctgctgggggattggggtctgttgggggctcggggcctgccagaggttcaggacctgccggggactcagggcctgctggaagttcaggacctgctggggatcagggcctgccagggatttagggtctgctgggcgggccaccttttggcctctccctcatgcttgaggccat

SEQ ID NO: 2 - DNA, 501 bp, Homo sapiens
gggcaacagagaaagattctatctcaaaaaaaaaaatttttttttaagttaaaaataaaataaagactttggggcaatacagggggtcctgggagtgtaacccataacccccaagagtgatttctgcaatctcgtttcaaattacagggtcaactgctatgatgtgtttggagcccagtcaccctttggtggctacaagatgtcggggagtggccgggagttgggcgagtacgggctgcaggcatacactaaagtgaaaactgtgagtgtgggacctgctgggggctcagggcctgttggggcttgagggtctgctggtggctcggagcctgctgggggattggggtctgttgggggctcggggcctgccagaggttcaggacctgccggggactcagggcctgctggaagttcaggacctgctggggatcagggcctgccagggatttagggtctgctgggcgggccaccttttggcctctccctcatgcttgaggccat

SEQ ID NO: 3 - DNA, 21 bp, Artificial Sequence
  probe
gttttcacttcagtgtatgcc

SEQ ID NO: 4 - DNA, 21 bp, Artificial Sequence
  probe
ggcatacactaaagtgaaaac

SEQ ID NO: 5 - DNA, 20 bp, Artificial Sequence
  probe
ttttcactttagtgtatgcc

SEQ ID NO: 6 - DNA, 19 bp, Artificial Sequence
  probe
cttcagtgtatgcctgcag

SEQ ID NO: 7 - DNA, 21 bp, Artificial Sequence
  primer
ctgggagtgtaacccataacc

SEQ ID NO: 8 - DNA, 20 bp, Artificial Sequence
  primer
cagcaggccctgagtccccg

SEQ ID NO: 9 - DNA, 50 bp, Artificial Sequence
  oligonucleotide
gggcgagtacgggctgcaggcatacactgaagtgaaaactgtgagtgtgg

SEQ ID NO: 10 - DNA, 50 bp, Artificial Sequence
  oligonucleotide
gggcgagtacgggctgcaggcatacactaaagtgaaaactgtgagtgtgg

SEQ ID NO: 11 - DNA, 50 bp, Artificial Sequence
  oligonucleotide
ccacactcacagttttcacttcagtgtatgcctgcagcccgtactcgccc

SEQ ID NO: 12 - DNA, 50 bp, Artificial Sequence
  oligonucleotide
ccacactcacagttttcactttagtgtatgcctgcagcccgtactcgccc

SEQ ID NO: 13 - DNA, 501 bp, Homo sapiens
agggctttagactgaataaccttggggataaactgaatctttcatatttaggaatagtagggattagtagcaaaaccctcaaatacattttagaaacacaatttcaggaatttgggtatgttaaattcatctagttacaatcttttctgaatctgaacagcttctctttattctgtagatggtggctgtaggaatctgtcgcacagatgaccacgtggttagtggcaakctggtgwccccccttcctgtgattttaggccatgaggcagccggcatcgtggagagtgttggagaaggggtgactacagtcaaaccaggtacaggattcacactcagggaacacgtgtggttcaccatccaggatttcccagcctggataaggaaaccaaggcaatgagagacgaaagtgcttgcacaaggtcaccgcgcagccaggacttcaggagtttcctctttccctctctgcctgcctgacccaagcatgtatgcattcaggcactc

SEQ ID NO: 14 - DNA, 501 bp, Homo sapiens
agggctttagactgaataaccttggggataaactgaatctttcatatttaggaatagtagggattagtagcaaaaccctcaaatacattttagaaacacaatttcaggaatttgggtatgttaaattcatctagttacaatcttttctgaatctgaacagcttctctttattctgtagatggtggctgtaggaatctgtcacacagatgaccacgtggttagtggcaakctggtgwccccccttcctgtgattttaggccatgaggcagccggcatcgtggagagtgttggagaaggggtgactacagtcaaaccaggtacaggattcacactcagggaacacgtgtggttcaccatccaggatttcccagcctggataaggaaaccaaggcaatgagagacgaaagtgcttgcacaaggtcaccgcgcagccaggacttcaggagtttcctctttccctctctgcctgcctgacccaagcatgtatgcattcaggcactc

SEQ ID NO: 15 - DNA, 18 bp, Artificial Sequence
  probe
tctgtcgcacngatgacc

SEQ ID NO: 16 - DNA, 18 bp, Artificial Sequence
  probe
tctgtcgcacggatgacc

SEQ ID NO: 17 - DNA, 18 bp, Artificial Sequence
  probe
tctgtcacacagatgacc

SEQ ID NO: 18 - DNA, 18 bp, Artificial Sequence
  probe
tgtcacacagatgaccac

SEQ ID NO: 19 - DNA, 46 bp, Artificial Sequence
  primer
gaaacacaatttcaggaatttgggtatgttaaattcatctagttac

SEQ ID NO: 20 - DNA, 25 bp, Artificial Sequence
  primer
ggwcaccagkttgccactaaccacg

SEQ ID NO: 21 - DNA, 50 bp, Artificial Sequence
  oligonucleotide
ggtggctgtaggaatctgtcgcacagatgaccacgtggttagtggcaacc

SEQ ID NO: 22 - DNA, 50 bp, Artificial Sequence
  oligonucleotide
ggtggctgtaggaatctgtcacacagatgaccacgtggttagtggcaacc

SEQ ID NO: 23 - DNA, 50 bp, Artificial Sequence
  oligonucleotide
ggttgccactaaccacgtggtcatctgtgcgacagattcctacagccacc

SEQ ID NO: 24 - DNA, 50 bp, Artificial Sequence
  oligonucleotide
ggttgccactaaccacgtggtcatctgtgtgacagattcctacagccacc

(19) United States
(12) Patent Application Publication
(10) Pub. No.: US 2012/0276533 A1
(43) Pub. Date: Nov. 1, 2012

(54) Method for Simultaneously Detecting Polymorphisms of Acetaldehyde Dehydrogenase 2 and Alcohol Dehydrogenase 2

(75) Inventors: Mitsuharu Hirai, Kyoto (JP); Aki Iguchi, Kyoto (JP)

(73) Assignee: ARKRAY, Inc., Kyoto (JP)

(21) Appl. No.: 13/458,735

(22) Filed: Apr. 27, 2012

(30) Foreign Application Priority Data
Apr. 28, 2011 (JP) 2011-102333

(51) Int. Cl. G01N 21/75; C12Q 1/68; C07H 21/04

(52) U.S. Cl. 435/6.11; 536/24.31

(57) ABSTRACT

A probe for detection of at least 1 type of genetic polymorphism of the ALDH2 gene rs671 and the ADH2 gene rs1229984, a kit therefore, and methods of detecting the polymorphism(s).

### CROSS-REFERENCE TO RELATED APPLICATION

This application claims priority from Japanese Patent Application No. 2011-102333 filed on Apr. 28, 2011, the entire subject matter of which is incorporated herein by reference in its entirety.

### SEQUENCE LISTING SUBMISSION VIA EFS-WEB

A computer readable text file, entitled “SequenceListing.txt,” created on or about Apr. 27, 2012 with a file size of about 2 kb contains the sequence listing for this application and is hereby incorporated by reference in its entirety.

### TECHNICAL FIELD

The present invention relates to probes which detect a polymorphism(s) in acetaldehyde dehydrogenase 2 (ALDH2) and alcohol dehydrogenase 2 (ADH2), a kit therefore, and methods of detecting the polymorphism(s) thereof.

### BACKGROUND ART

Representative examples of enzymes involved in alcohol metabolism include ADH (alcohol dehydrogenase) and ALDH (aldehyde dehydrogenase). ADH metabolizes ethanol to acetaldehyde, and ALDH metabolizes acetaldehyde to acetic acid.

There are 3 types of ADH, that is, ADH1, ADH2 and ADH3, and, as genetic polymorphisms involved in phenotypes of the ADH2 gene, there are ADH2*1 (WT), ADH2*2 and ADH2*3 (F Tanaka et al., Hepatology, Volume 23, Issue 2, pages 234-239, February 1996). In Japanese, their frequencies are *1/*1: 8.4%, *1/*2: 34.9% and *2/*2: 56.7%, and *3 hardly exists (Kyoko Saito et al., Do ethanol metabolic enzymes modify the relationship between alcohol drinking and the blood pressure? 15th Meeting of Society of Blood Pressure Control, Subject 3(healthcare.omron.co.jp/medical/study/pdf/past15_{—}03.pdf)).

As genetic polymorphisms involved in phenotypes of the ALDH2 gene, there are *1(WT) and *2 (F Tanaka et al., Hepatology, Volume 23, Issue 2, pages 234-239, February 1996), and their frequencies in Japanese are *1/*1: 52.8%, *1/*2: 40.9% and *2/*2: 6.3% (Kyoko Saito et al., Do ethanol metabolic enzymes modify the relationship between alcohol drinking and the blood pressure? 15th Meeting of Society of Blood Pressure Control, Subject 3).

Among these genetic polymorphisms, ADH2*2 and ALDH2 are reported to be involved in alcohol dependence, liver diseases and liver cancer (F Tanaka et al., Hepatology, Volume 23, Issue 2, pages 234-239, February 1996; Keitaro Matsuo et al., Research Report of The Uehara Memorial Foundation, 23 (2009), Molecular Epidemiologic Research for Evaluation of Influences of the Genetic Background and the Drinking Habit on the Risk of Developing Pancreatic Cancer (http://ueharazaidan.yoshida-p.net/houkokushu/Vol.23/pdf/046_report.pdf)).

CYP2E1 is also reported to have the same function as ADH2 and ALDH2 (JP 2008-79604 A).

JP 2008-79604 A describes a primer set and a probe set for detection of genetic (the ALDH2, ADH2 and CYP2E1 genes) mutations involved in the capacity to metabolize alcohol and the alcohol tolerance, by hybridization. However, there are problems in that (1) the detection needs to be carried out using one reaction system (one tube) per one mutation involved in alcohol metabolism or the like, which is laborious, costly and time-consuming; (2) since DNA purified from saliva/whole blood needs to be used, much labor, cost and time are required, so that the measurement may not be simply carried out; and (3) since an amplification product needs to be handled for microarray analysis, there is the risk of contamination of the amplification product to another sample.

On the other hand, there are known methods wherein a region containing a mutation is amplified by PCR and a fluorescently labeled nucleic acid probe is used to carry out melting curve analysis, followed by analyzing the mutation based on the result of the melting curve analysis (JP 2001-286300 A and JP 2002-119291 A). However, these literatures only teach that the probe is designed such that, when a quenching probe labeled at its end with a fluorescent dye is hybridized with a target nucleic acid, a plurality of base pairs of the probe-nucleic acid hybrid form at least one GC pair at the end. Further, these methods had a problem in that the methods are not necessarily applicable to an arbitrary sequence.

### SUMMARY OF THE INVENTION

The present invention aims to specify probes effective for detecting rs671, which is a genetic polymorphism of acetaldehyde dehydrogenase 2 (ALDH2), and rs1229984, which is a genetic polymorphism of alcohol dehydrogenase 2 (ADH2), and to provide a method for detecting these genetic polymorphisms at the same time and a kit therefore.

The present inventors discovered that, by designing probes based on specific regions containing the polymorphism of the acetaldehyde dehydrogenase 2 (ALDH2) gene rs671 and the polymorphism of the alcohol dehydrogenase 2 (ADH2) gene rs1229984 and carrying out melting curve analysis using the probes, the mutations may be detected, thereby completing the present invention.

That is, the present invention in one aspect includes a labeled probe comprising at least one oligonucleotide selected from the group consisting of oligonucleotides (P1), (P2), and (P3):

(P1) an oligonucleotide comprising a sequence at least about 85% identical to a complementary nucleotide sequence of 11 to 50 nucleotides to nucleotides 241 to 251 of SEQ ID NO:1 or 2;

(P2) an oligonucleotide comprising a sequence at least about 85% identical to a complementary nucleotide sequence of 11 to 50 nucleotides to nucleotides 251 to 261 of SEQ ID NO:1 or 2; and

(P3) an oligonucleotide comprising a sequence at least about 85% identical to a complementary nucleotide sequence of 12 to 50 nucleotides to nucleotides 201 to 212 of SEQ ID NO:13.

In some embodiments, in said oligonucleotide (P1), the nucleotide corresponding to the nucleotide at position 241 is cytosine labeled with a fluorescent dye; in said oligonucleotide (P2), the nucleotide corresponding to the nucleotide at position 261 is cytosine labeled with a fluorescent dye; and in said oligonucleotide (P3), the nucleotide corresponding to the nucleotide at position 212 is cytosine labeled with a fluorescent dye.

The present invention in another aspect includes a probe which detects a polymorphism(s) of the ALDH2 gene rs671 and the ADH2 gene rs1229984, comprising at least one of fluorescently labeled oligonucleotide selected from (P1) to (P3′) below:

(P1) an oligonucleotide comprising a nucleotide sequence complementary to a nucleotide sequence of 11 to 50 consecutive nucleotides containing nucleotides 241 to 251 in SEQ ID NO:1 or 2 or a homologous sequence thereof, wherein the nucleotide corresponding to the nucleotide at position 241 is cytosine labeled with a fluorescent dye;

(P1′) an oligonucleotide comprising a nucleotide sequence which hybridizes with a nucleotide sequence of 11 to 50 consecutive nucleotides containing nucleotides 241 to 251 in SEQ ID NO:1 or 2 under stringent conditions, wherein the nucleotide corresponding to the nucleotide at position 241 is cytosine labeled with a fluorescent dye;

(P2) an oligonucleotide comprising a nucleotide sequence of 11 to 50 consecutive nucleotides containing nucleotides 251 to 261 in SEQ ID NO:1 or 2 or a homologous sequence thereof, wherein the nucleotide corresponding to the nucleotide at position 261 is cytosine labeled with a fluorescent dye;

(P2′) an oligonucleotide comprising a nucleotide sequence of 11 to 50 consecutive nucleotides containing nucleotides 251 to 261 in SEQ ID NO:1 or 2 or a nucleotide sequence which hybridizes with the complementary strand of SEQ ID NO: 1 or 2 under stringent conditions, wherein the nucleotide corresponding to the nucleotide at position 261 is cytosine labeled with a fluorescent dye;

(P3) an oligonucleotide comprising a nucleotide sequence of 12 to 50 consecutive nucleotides containing nucleotides 201 to 212 in SEQ ID NO:13 or a homologous sequence thereof, wherein the nucleotide corresponding to the nucleotide at position 212 is cytosine labeled with a fluorescent dye; and

(P3′) an oligonucleotide comprising a nucleotide sequence of 12 to 50 consecutive nucleotides containing nucleotides 201 to 212 in SEQ ID NO:13 or a nucleotide sequence which hybridizes with the complementary strand of SEQ ID NO:13 under stringent conditions, wherein the nucleotide corresponding to the nucleotide at position 212 is cytosine labeled with a fluorescent dye.

In another aspect, oligonucleotides (P1) and (P1′) described herein have the nucleotide corresponding to the nucleotide at position 241 labeled with a fluorescent dye at the first, second or third position from the 3′ end; oligonucleotides (P2) and (P2′) described herein have the nucleotide corresponding to the nucleotide at position 261 labeled with a fluorescent dye at the first, second or third position from the 3′ end; and oligonucleotides (P3) and (P3′) described herein have the nucleotide corresponding to the nucleotide at position 212 labeled with a fluorescent dye at the first, second or third position from the 3′ end.

In yet another aspect, oligonucleotides (P1) and (P1′) described herein have the nucleotide corresponding to the nucleotide at position 241 labeled with a fluorescent dye at the 3′ end; oligonucleotides (P2) and (P2′) described herein have the nucleotide corresponding to the nucleotide at position 261 labeled with a fluorescent dye at the 3′ end; and oligonucleotides (P3) and (P3′) described herein have the nucleotide corresponding to the nucleotide at position 212 labeled with a fluorescent dye at the 3′ end.

In a further aspect, oligonucleotide according to some embodiments of the present invention emits fluorescence when the oligonucleotide is not hybridized with a target sequence, and the fluorescence intensity decreases or increases when the oligonucleotide is hybridized with the target sequence.

In a yet further aspect, the fluorescence intensity decreases when said oligonucleotide is hybridized with the target sequence.

In another aspect, oligonucleotides (P1) to (P3′) may have 12 to 30 consecutive nucleotides, 15 to 30 consecutive nucleotides, or 18 to 30 consecutive nucleotides.

In another aspect, the probes described herein are probes for melting curve analysis.

According to some embodiments of the present invention, the method for detecting at least one polymorphism selected from the group consisting of the polymorphism of the ALDH2 gene rs671 and the polymorphism of the ADH2 gene rs1229984, by using the probe for detecting a polymorphism as described herein.

In one aspect, the method for detecting a polymorphism(s) described herein comprises:

(I) bringing the probe for detection of a polymorphism(s) described herein into contact with single-stranded nucleic acid in a sample, to allow hybridization of said fluorescently labeled oligonucleotide(s) with said single-stranded nucleic acid, thereby obtaining a hybrid-forming body/bodies;

(II) changing the temperature of the sample containing the hybrid-forming body/bodies to dissociate the hybrid-forming body/bodies, and measuring fluctuation of a fluorescence signal(s) due to the dissociation of the hybrid-forming body/bodies;

(III) determining the Tm value(s), which is/are the dissociation temperature(s) of the hybrid-forming body/bodies, based on the fluctuation of said signal(s); and

(IV) determining based on said Tm value(s) the presence of at least one polymorphism, or the abundance ratio(s) of a nucleic acid(s) having a polymorphism(s), which polymorphism(s) is/are at least one polymorphism selected from the group consisting of the polymorphism of the ALDH2 gene rs671 and the polymorphism of the ADH2 gene rs1229984.

In another aspect, the method for detecting a polymorphism(s) described herein comprises amplifying nucleic acid before said Step (I) or at the same time with said Step (I)

The method according to some embodiments of the present invention comprises detecting at least one polymorphism selected from the group consisting of the polymorphism of the ALDH2 gene rs671 and the polymorphism of the ADH2 gene rs1229984, by the method for detecting a polymorphism(s) described herein; and evaluating capacity to metabolize, and/or judging tolerance to, alcohol based on the presence/absence of said polymorphism(s).

(14) The kit according to additional embodiments of the present invention comprises the probe for detection of a polymorphism(s) described herein, in which kit is used for detecting at least one polymorphism selected from the group consisting of genetic polymorphisms of the ALDH2 gene and genetic polymorphisms of the ADH2 gene.

In one aspect, the kit for detection of a polymorphism(s) described herein further comprises a primer that enables amplification using as a template a region in the nucleotide sequence shown in SEQ ID NO:1 or 2 comprising a sequence with which said oligonucleotide (P1), (P1′), (P2) or (P2′) hybridizes, and a primer that enables amplification using as a template a region in the nucleotide sequence shown in SEQ ID NO:13 comprising a sequence with which said oligonucleotide (P3) or (P3′) hybridizes.

The method according to some embodiments of the present invention uses the probe described herein, and primers which are used for detecting at least one polymorphism selected from the group consisting of the polymorphism of the ALDH2 gene rs671 and the polymorphism of the ADH2 gene rs1229984, said primers comprising the oligonucleotides (P4) and (P5) and/or (P6) and (P7) described below:

(P4) an oligonucleotide comprising a nucleotide sequence of 21 to 60 consecutive nucleotides containing nucleotides 89 to 109 in SEQ ID NO:1 or 2;

(P5) an oligonucleotide comprising a nucleotide sequence complementary to a nucleotide of 20 to 60 consecutive nucleotides containing nucleotides 388 to 407 in SEQ ID NO:1 or 2;

(P6) an oligonucleotide comprising a nucleotide sequence of 46 to 60 consecutive nucleotides containing nucleotides 93 to 138 in SEQ ID NO:13; and

(P7) an oligonucleotide comprising a nucleotide sequence complementary to a nucleotide of 25 to 60 consecutive nucleotides containing nucleotides 214 to 238 in SEQ ID NO:13.

The method according to some embodiments of the present invention for evaluating capacity to metabolize, and/or judging tolerance to, alcohol, comprising evaluating capacity to metabolize, and/or judging tolerance to, alcohol based on the presence/absence of a polymorphism(s) comprises detecting at least one polymorphism selected from the group consisting of the polymorphism of the ALDH2 gene rs671 and the polymorphism of the ADH2 gene rs1229984 by the method according to (16), and evaluating capacity to metabolize, and/or judging tolerance to, alcohol based on the presence/absence of the polymorphism(s).

The reagent kit according to some embodiments of the present invention for detection of at least one polymorphism selected from the group consisting of the polymorphism of the ALDH2 gene rs671 and the polymorphism of the ADH2 gene rs1229984 comprises the probe described herein and primers comprising the oligonucleotides (P4) and (P5) and/or (P6) and (P7) described herein.

With the probes of the present invention, the polymorphism of the acetaldehyde dehydrogenase 2 (ALDH2) gene rs671 and the polymorphism of the alcohol dehydrogenase 2 (ADH2) gene rs1229984 may be detected clearly at the same time. In view of clinical necessity to confirm gene mutations of ALDH2 and ADH2, being able to detect the two mutations at the same time is of significance.

By just adding the probes of the present invention and carrying out melting curve analysis (Tm analysis), the genetic polymorphisms of ALDH2 and ADH2 may be detected at the same time.

Since, by using the method of the present invention, the operation of recovery of an amplification product may be eliminated even in cases where PCR is carried out, there is hardly the risk of contamination. Further, since the operations in the method of the present invention are simple, they may be easily automated.

In the detection method of the present invention, nucleic acid originally contained in unpurified saliva/whole blood may be used as a template.

### BRIEF DESCRIPTION OF THE DRAWINGS

FIG. 1 shows the relationship between the amount of change in the fluorescence intensity of TAMRA per unit time (d the amount of increase in the fluorescence intensity/t) and the temperature in the Tm analysis in Example 1 in which 3T-ALDH2*2-wt-R1-21 was used as a probe, ALDH2*1 F50 (wild type) and ALDH2*2 F50 (mutant type) were used as templates, and TAMRA was used as a fluorescent dye. The ordinate indicates the amount of change in the fluorescence intensity per unit time (d the amount of increase in the fluorescence intensity/t), and the abscissa indicates the temperature (° C.). In the figures below, results obtained by the same experiments using different combinations of templates and a fluorescent dye are shown.

FIG. 2 shows the result obtained in Example 1 by using 3T-ALDH2*2-mt-F1-21 as a probe, ALDH2* 1 R50 (wild type) and ALDH2*2 R50 (mutant type) as templates, and TAMRA as a fluorescent dye.

FIG. 3 shows the result obtained in Example 1 by using 3T-ALDH2*2-wt-R3-20 as a probe, ALDH2* 1 F50 (wild type) and ALDH2*2 F50 (mutant type) as templates, and TAMRA as a fluorescent dye.

FIG. 4 shows the result obtained in Comparative Example 1 by using 5T-ALDH2*2-wt-R2-19 as a probe, ALDH2* 1 F50 (wild type) and ALDH2*2 F50 (mutant type) as templates, and TAMRA as a fluorescent dye.

FIG. 5A shows the result obtained in Example 2 by using 3FL-ADH2-WT-F3 as a probe, ADH2*1-R50 (wild type) and ADH2*2-R50 (mutant type) as templates, and BODIPY FL as a fluorescent dye.

FIG. 5B shows a diagram in which the amount of change in the fluorescence intensity in FIG. 5A is shown at an enlarged scale.

FIG. 6A shows the result obtained in Comparative Example 2 by using 3FL-ADH2-mt-F1 as a probe, ADH2*1-R50 (wild type) and ADH2*2-R50 (mutant type) as templates, and BODIPY FL as a fluorescent dye.

FIG. 6B shows a diagram in which the amount of change in the fluorescence intensity in FIG. 6A is shown at an enlarged scale.

FIG. 7A shows the result obtained in Comparative Example 2 by using 3FL-ADH2-mt-F2 as a probe, ADH2*1-R51 (wild type) and ADH2*2-R51 (mutant type) as templates, and BODIPY FL as a fluorescent dye.

FIG. 7B shows a diagram in which the amount of change in the fluorescence intensity in FIG. 7A is shown at an enlarged scale.

FIG. 8A shows the result obtained in Example 3 by using 3T-ALDH2*2-mt-F1-21 as a probe, an oral swab as a template, and TAMRA as a fluorescent dye.

FIG. 8B shows the result obtained in Example 3 by using 3FL-ADH2-WT-F3 as a probe, an oral swab as a sample, and BODIPY FL as a fluorescent dye.

FIG. 9A shows the result obtained in Example 3 by using 3T-ALDH2*2-mt-F1-21 as a probe, whole blood as a sample, and TAMRA as a fluorescent dye.

FIG. 9B shows the result obtained in Example 3 by using 3FL-ADH2-WT-F3 as a probe, whole blood as a sample, and BODIPY FL as a fluorescent dye.

FIG. 10A shows the result obtained in Example 3 by using 3T-ALDH2*2-mt-F1-21 as a probe, purified DNA as a sample, and TAMRA as a fluorescent dye.

FIG. 10B shows the result obtained in Example 3 by using 3FL-ADH2-WT-F3 as a probe, purified DNA as a sample, and BODIPY FL as a fluorescent dye.

### DESCRIPTION OF EMBODIMENTS

### <1> Probe of Present Invention and Detection Method of Present Invention

The probe according to some embodiments of the present invention is a labeled probe comprising at least one oligonucleotide selected from the group consisting of oligonucleotides (P1), (P1′), (P2), (P2′), (P3), and (P3′) described herein. In one aspect, the probes are for detecting a polymorphism(s) in ALDH2 gene rs671 and ADH2 gene rs1229984. In another aspect, the probes are fluorescently labeled.

The oligonucleotide (P1) may comprise a nucleotide sequence complementary to a nucleotide sequence of 11 to 50 consecutive nucleotides containing nucleotides 241 to 251 in SEQ ID NO:1 or 2 or a homologous sequence thereof, wherein the nucleotide corresponding to the nucleotide at position 241 is cytosine labeled with a fluorescent dye;

The oligonucleotide (P1′) may comprise a nucleotide sequence which hybridizes with a nucleotide sequence of 11 to 50 consecutive nucleotides containing nucleotides 241 to 251 in SEQ ID NO:1 or 2 under stringent conditions, wherein the nucleotide corresponding to the nucleotide at position 241 is cytosine labeled with a fluorescent dye;

The oligonucleotide (P2) may comprise a nucleotide sequence of 11 to 50 consecutive nucleotides containing nucleotides 251 to 261 in SEQ ID NO:1 or 2 or a homologous sequence thereof, wherein the nucleotide corresponding to the nucleotide at position 261 is cytosine labeled with a fluorescent dye;

The oligonucleotide (P2′) may comprise a nucleotide sequence of 11 to 50 consecutive nucleotides containing nucleotides 251 to 261 in SEQ ID NO:1 or 2 or a nucleotide sequence which hybridizes with the complementary strand of SEQ ID NO: 1 or 2 under stringent conditions, wherein the nucleotide corresponding to the nucleotide at position 261 is cytosine labeled with a fluorescent dye;

The oligonucleotide (P3) may comprise a nucleotide sequence of 12 to 50 consecutive nucleotides containing nucleotides 201 to 212 in SEQ ID NO:13 or a homologous sequence thereof, wherein the nucleotide corresponding to the nucleotide at position 212 is cytosine labeled with a fluorescent dye; and

The oligonucleotide (P3′) may comprise a nucleotide sequence of 12 to 50 consecutive nucleotides containing nucleotides 201 to 212 in SEQ ID NO:13 or a nucleotide sequence which hybridizes with the complementary strand of SEQ ID NO:13 under stringent conditions, wherein the nucleotide corresponding to the nucleotide at position 212 is cytosine labeled with a fluorescent dye.

In additional embodiments, the oligonucleotide (P1) may comprise or consists of a sequence at least about 85%, 86%, 87%, 88%, 89%, 90%, 91%, 92%, 93%, 94%, 95%, 96%, 97%, 98%, 99%, or 100% identical to a complementary nucleotide sequence of 11 to 50 nucleotides to nucleotides 241 to 251 of SEQ ID NO:1 or 2; the oligonucleotide (P2) may comprise or consists of a sequence at least about 85%, 86%, 87%, 88%, 89%, 90%, 91%, 92%, 93%, 94%, 95%, 96%, 97%, 98%, 99%, or 100% identical to a complementary nucleotide sequence of 11 to 50 nucleotides to nucleotides 251 to 261 of SEQ ID NO:1 or 2; and the oligonucleotide (P3) may comprise or consists of a sequence at least about 85%, 86%, 87%, 88%, 89%, 90%, 91%, 92%, 93%, 94%, 95%, 96%, 97%, 98%, 99%, or 100% identical to a complementary nucleotide sequence of 12 to 50 nucleotides to nucleotides 201 to 212 of SEQ ID NO:13.

In further embodiments, the oligonucleotide (P1) comprises a complementary nucleotide sequence of 11 to 50 nucleotides to nucleotides 241 to 251 of SEQ ID NO:1 or 2; the oligonucleotide (P2) comprises a complementary nucleotide sequence of 11 to 50 nucleotides to nucleotides 251 to 261 of SEQ ID NO:1 or 2; and the oligonucleotide (P3) comprises a complementary nucleotide sequence of 12 to 50 nucleotides to nucleotides 201 to 212 of SEQ ID NO:13.

The polymorphism of the ALDH2 gene rs671 herein is located at nucleotide position 251 of SEQ ID NOs: 1 and 2. The polymorphism of the ADH2 gene rs1229984 is located at nucleotide position 201 of SEQ ID NOs:13 and 14. These rs numbers indicate registration numbers for the dbSNP database by National Center for Biotechnology Information (ncbi.nlm.nih gov/projects/SNP).

The probe described herein may be prepared in the same manner as the probes described in JP 2001-286300 A and JP 2002-119291 A except that the probe described herein has the above-described specified sequence in the nucleotide sequence shown in SEQ ID NO:1 (sequence having the wild-type nucleotide of the ALDH2 gene) or the nucleotide sequence shown in SEQ ID NO:2 (sequence having the mutant-type (polymorphic) nucleotide of the ALDH2 gene), and the above-described specified sequence in the nucleotide sequence shown in SEQ ID NO:13 (sequence having the wild-type nucleotide of the ADH2 gene).

The term “homologous sequence” or “identical sequence” herein means that a nucleotide sequence comprises a sequence having an identity of 80%, 85%, 86%, 87%, 88%, 89%, 90%, 91%, 92%, 93%, 94%, 95%, 96%, 97%, 98% or 99% to the complementary strand of a particular nucleotide sequence. In the present invention, 100% identity may be included. The hybridization herein may be carried out according to a known method or a method corresponding thereto, such as the method described in Molecular Cloning 3rd (J. Sambrook et al., Cold Spring Harbor Lab. Press, 2001). This literature is hereby incorporated in the present specification by reference.

The stringent conditions mean conditions under which a specific hybrid is formed while nonspecific hybrids are not formed. Typical examples of the stringent conditions include conditions under which hybridization is performed with a potassium concentration of about 25 mM to about 50 mM and a magnesium concentration of about 1.0 mM to about 5.0 mM. Examples of the conditions in the present invention include conditions under which hybridization is performed in Tris-HCl (pH 8.6), 25 mM KCl and 1.5 mM MgCl₂, but the conditions are not limited thereto. Other examples of the stringent conditions include those described in Molecular Cloning 3rd (J. Sambrook et al., Cold Spring Harbor Lab. Press, 2001). This literature is hereby incorporated in the present specification by reference. Those skilled in the art may easily select such conditions by controlling the hybridization reaction and/or changing the salt conditions of the hybridization reaction solution.

Oligonucleotides described herein may include modified oligonucleotides. As a unit of the oligonucleotides, ribonucleotides, deoxylibonucleotides, and artificial nucleic acids may be included. The artificial nucleic acids may include DNA, RNA, RNA analogue LNA (Locked Nucleic Acid); PNA (Peptide Nucleic Acid); BNA (Bridged Nucleic Acid) etc. The above-mentioned oligonucleotides may be comprised of one or more kinds of the units.

In one aspect, the probes (P1) and (P1′) of the present invention may have a sequence complementary to a nucleotide sequence of 11 to 50 consecutive nucleotides containing nucleotides 241 to 251 in SEQ ID NO:1 or 2 or a homologous sequence thereof. That is, the sequence of the probes (P1) and (P1′) of the present invention may be complementary to a nucleotide sequence of 11 to 50 consecutive nucleotides containing nucleotides 241 to 251, but the sequence of the probe does not need to be completely complementary to such a nucleotide sequence. Further, the nucleotide corresponding to the nucleotide at position 241 may be cytosine and labeled with a fluorescent dye.

In another aspect, the probes (P2) and (P2′) of the present invention may have a sequence of 11 to 50 consecutive nucleotides containing nucleotides 251 to 261 in SEQ ID NO:1 or 2 or a homologous sequence thereof. That is, the sequence of probes (P2) and (P2′) of the present invention may be homologous to a nucleotide sequence of 11 to 50 consecutive nucleotides containing nucleotides 251 to 261, but the sequence of the probe does not need to be completely identical to such a nucleotide sequence. Further, the nucleotide corresponding to the nucleotide at position 261 may be cytosine and labeled with a florescent dye.

In another aspect, the probes (P3) and (P3′) of the present invention may have a sequence of 12 to 50 consecutive nucleotides containing nucleotides 201 to 212 in SEQ ID NO:13 or a homologous sequence thereof. That is, the sequence of probes (P3) and (P3′) of the present invention may be homologous to a nucleotide sequence of 12 to 50 consecutive nucleotides containing nucleotides 201 to 212, but the sequence of the probe does not need to be completely identical to such a nucleotide sequence. Further, the nucleotide corresponding to the nucleotide at position 212 may be cytosine and labeled with a fluorescent dye.

In another aspect, the nucleotide length of each of the probes (P1) to (P3′) of the present invention may be 11 to 50, 12 to 30, 15 to 30, or 18 to 30, for example.

Examples of the nucleotide sequence of the probe to be used in the present invention for detecting the polymorphism of the ALDH2 gene rs671 include 5′-gttttcacttCagtgtatgcc-3′ (SEQ ID NO:3), 5′-ggcatacactAaagtgaaaac-3′ (SEQ ID NO:4) and 5′-ttttcacttTagtgtatgcc-3′ (SEQ ID NO:5). Each nucleotide indicated by an uppercase letter represents the position of mutation.

Examples of the nucleotide sequence of the probe for detecting the polymorphism of the ADH2 gene rs1229984 include 5′-TCTGTCNCACGGATGACC-3′ (SEQ ID NO:15). In this sequence, “N” represents a, t, g or c. More particularly, the nucleotide sequence is 5′-TCTGTCGCACGGATGACC-3′ (SEQ ID NO:16).

Examples of the fluorescent dye which may be used include those described in JP 2001-286300 A and JP 2002-119291 A, and specific examples of the fluorescent dye include PACIFIC BLUE (trademark), FAM (trademark), TAMRA (trademark) and BODIPY (trademark) FL. Examples of the method for binding the fluorescent dye to the oligonucleotide include conventional methods such as the methods described in JP 2001-286300 A and JP2002-119291 A.

For example, the probe according to some embodiments of the present invention emits fluorescence from a fluorescent dye when the probe is not hybridized with the target sequence, and the fluorescence from the fluorescent dye decreases or increases when the probe is hybridized with the target sequence. For example, the probe according to additional embodiments of the present invention is a quenching probe which emits fluorescence from a fluorescent dye when the probe is not hybridized, and the fluorescence from the fluorescent dye is quenched when the probe is hybridized.

Further, the probe according to further embodiments of the present invention has a nucleotide labeled with a fluorescent dye at the first, second or third position from the 3′ end, or the probe has the 3′ end which is labeled with a fluorescent dye, for example.

The nucleotide labeled with a fluorescent dye in the probe described herein is the nucleotide at position 241 or 261 in SEQ ID NO:1 or 2. In SEQ ID NO:13 or 14, the nucleotide at position 212 is labeled.

The detection method of the present invention uses the probe described herein and comprises

(I) bringing the probe described herein for detecting a polymorphism into contact with single-stranded nucleic acid in a sample, to allow hybridization of the fluorescently labeled oligonucleotide(s) with the single-stranded nucleic acid, thereby obtaining a hybrid-forming body/bodies;

(II) changing the temperature of the sample containing the hybrid-forming body/bodies to dissociate the hybrid-forming body/bodies, and measuring fluctuation of a fluorescence signal(s) due to the dissociation of the hybrid-forming body/bodies;

(III) determining the Tm value(s), which is/are the dissociation temperature(s) of the hybrid-forming body/bodies, based on the fluctuation of the signal(s); and

(IV) determining based on the Tm value(s) the presence of at least one polymorphism, or the abundance ratio(s) of a nucleic acid(s) having a polymorphism(s), which polymorphism(s) is/are at least one polymorphism selected from the group consisting of the polymorphism of the ALDH2 gene rs671 and the polymorphism of the ADH2 gene rs1229984.

The detection method of the present invention may be carried out according to conventional methods for nucleic acid amplification and melting curve analysis (Tm analysis) except that the probe described herein is used. Further, the detection method of the present invention may also comprise amplifying nucleic acid before the Step (I) or at the same time with the Step (I).

The method of amplification may use a polymerase, and examples of the method include PCR, IMAY and LAMP. When the amplification is carried out by a method using a polymerase, the amplification may be carried out in the presence of the probe described herein. Those skilled in the art may easily control reaction conditions and the like of the amplification depending on the probe to be used. By this, the detection may be carried out just by analyzing the Tm value of the probe after the amplification of nucleic acid, so that the amplification product does not need to be subjected to purification and/or the like after the reaction. Therefore, there is no risk of contamination by an amplified product. Further, since the detection may be carried out with the same apparatus as the one necessary for the amplification, it is not necessary even to transfer the container. Therefore, automation may also be easily done.

In the present invention, the DNA in the sample may be either a single-stranded DNA or a double-stranded DNA. In cases where the DNA is a double-stranded DNA, for example, the step of dissociating the double-stranded DNA in the sample by heating may be included before the hybridization step. By dissociating the double-stranded DNA into single-stranded DNAs, hybridization with the detection probe is made possible in the subsequent hybridization step.

In the present invention, the ratio (molar ratio) of the probe described herein to be added with respect to the DNA in the sample is not restricted, and the ratio is, for example, 1 or less, or 0.1 or less with respect to the DNA in the sample in view of securing a sufficient detection signal. In this case, for example, the DNA in the sample may be either the total of DNA having the polymorphism(s) to be detected and DNA which does not have the polymorphism(s) and should not be detected, or the total of an amplification product(s) containing a sequence(s) having the polymorphism(s) to be detected and amplification products containing sequences which do not have the polymorphism(s) to be detected and should not be detected. Although the ratio of the DNA to be detected in the DNA in the sample is usually not known, the ratio (molar ratio) of the probe to be added with respect to the DNA to be detected (the amplification product(s) containing the sequence(s) to be detected), as a result, is 10 or less, 5 or less, or 3 or less, for example. The lower limit of the ratio is not restricted, and the ratio is, for example, 0.001 or more, 0.01 or more, or 0.1 or more. The ratio of the probe described herein to be added with respect to the DNA may be, for example, either the molar ratio with respect to the double-stranded DNA or the molar ratio with respect to the single-stranded DNA.

Determination of the Tm value will now be described. Heating a solution containing double-stranded DNA causes an increase in the absorbance at 260 nm. This is caused because hydrogen bonds between the both strands of the double-stranded DNA are unraveled by the heat and the double-stranded DNA is dissociated into single-stranded DNAs (melting of DNA). When all the double-stranded DNAs are dissociated into single-stranded DNAs, the absorbance becomes about 1.5 times as large as that observed when the heating was started (absorbance by only double-stranded DNA), and, by this, completion of the melting may be judged. Based on this phenomenon, the melting temperature Tm may be generally defined as the temperature at which increase in the absorbance reached 50% of the total increase in the absorbance.

In the present invention, measurement of the signal fluctuation due to the temperature change for determination of the Tm value may be carried out also by measuring the absorbance at 260 rim based on the above-mentioned principle, but the measurement may be carried out based on a signal from a label added to the probe described herein, which signal fluctuates depending on the state of hybrid formation between the DNA and the probe. Therefore, as the probe described herein, the above-mentioned labeled probe may be used. Examples of the labeled probe include a fluorescently labeled oligonucleotide probe which emits fluorescence when it is not hybridized with the target sequence and whose fluorescence intensity decreases (quenching) when the probe is hybridized with the target sequence, and a fluorescently labeled oligonucleotide probe which emits fluorescence when it is not hybridized with the target sequence and whose fluorescence intensity increases when the probe is hybridized with the target sequence. In the case of the former probe, the probe shows no signal or a weak signal when it is forming a hybrid (double-stranded DNA) with the sequence to be detected, while the probe shows a signal or the signal increases when the probe is released by heating. In the case of the latter probe, the probe shows a signal by forming a hybrid (double-stranded DNA) with the sequence to be detected, while the signal decreases (disappears) when the probe is released by heating. Therefore, by detecting such a change in the signal due to the label under conditions (with the absorbance or the like) specific to the signal, determination of the progress of melting and the Tm value may be carried out similarly to the case of measurement of the absorbance at 260 nm. For example, the labeling substance in the labeled probe is as mentioned above, and the probe may be a fluorescent dye-labeled probe.

The nucleic acid to be used as a template for carrying out the nucleic acid amplification is not restricted as long as it contains a nucleic acid, and examples of the nucleic acid include those derived from, or those which may be derived from, arbitrary biological origins such as blood; oral mucosal suspensions; somatic cells of nails, hairs and the like; germ cells; milks; ascitic fluids; paraffin-embedded tissues; gastric juices; fluids obtained by gastric lavage; peritoneal fluids; amniotic fluids; and cell cultures. The nucleic acid as a template may be used as it is directly after being obtained from the origin, or may be pretreated to modify properties of the sample before being used.

The method of nucleic acid amplification is further described by way of an example using PCR. The primer pair used in the PCR may be designed in the same manner as in the method for designing a primer pair for conventional PCR, except that the primer pair is designed such that a region with which the probe described herein may hybridize is amplified. The length and the Tm value of each primer is usually 12 mer to 40 mer and 40 to 70° C., or 16 mer to 30 mer and 55 to 65° C. The lengths of the primers of the primer pair may be different from each other, but the Tm values of the both primers may be almost the same (the difference is usually 2° C. or more). The Tm value is a value calculated by the Nearest Neighbor method. Examples of the primer pair include those composed of primers having nucleotide sequences selected from SEQ ID NOs:7, 8, 19 and 20.

The PCR may be carried out in the presence of the probe described herein. By this, the Tm analysis may be carried out without subjecting the amplified product to purification and/or the like after the amplification reaction. Those skilled in the art may easily control the Tm values of the primers and the reaction conditions for the PCR (the composition of the reagent, number of cycles, and the like) depending on the probe used.

The Tm analysis may be carried out in the same manner as in a conventional method except that fluorescence from the fluorescent dye of the probe described herein is measured. The measurement of fluorescence may be carried out using the excitation light having a wavelength dependent on the fluorescent dye, to measure the light having the emission wavelength. The heating rate in the Tm analysis is usually 0.1 to 1° C./second. The composition of the reaction solution used for carrying out the Tm analysis is not restricted as long as the probe may hybridize with a nucleic acid having the complementary sequence of the nucleotide sequence of the probe, and usually, the concentration of monovalent cations is 1.5 to 5 mM, and pH is 7 to 9. Since the reaction solution in an amplification method using a DNA polymerase, such as PCR, usually satisfies these conditions, the reaction solution after the amplification may be used as it is for the Tm analysis.

Detection of the polymorphism of the ALDH2 gene rs671 and the polymorphism of the ADH2 gene rs1229984 based on the result of the Tm analysis may be carried out according to a conventional method. The detection in the present invention includes detection of the presence/absence of a mutation and determination of the abundance of a nucleic acid having a polymorphism.

By the probe and the method for detecting a polymorphism of the present invention, polymorphisms in the ALDH2 gene and the ADH2 gene may be detected, and, based on the presence/absence of each polymorphism, the capacity to degrade acetaldehyde or alcohol may be predicted. More particularly, in cases where the nucleotides of the polymorphism of the ALDH2 gene rs671 are G/G, the gene is the wild type and hence suggested to have a high capacity to degrade acetaldehyde, while in cases where the nucleotides are G/A, which is the heterozygote, or A/A, which is the homozygote of the mutant base, the capacity to degrade acetaldehyde is suggested to be low; and in cases where the nucleotides of the polymorphism of the ADH2 gene rs1229984 are G/G, the gene is the wild type and hence suggested to have a low capacity to degrade alcohol, while in cases where the nucleotides are G/A, which is the heterozygote, or A/A, which is a homozygote, the capacity to degrade alcohol is suggested to be high.

Based on the result of prediction of the capacities to degrade acetaldehyde and alcohol, tolerance to alcohol and diseases related to the alcohol metabolism such alcohol dependence, liver diseases and liver cancer may be predicted.

Further, in the method of the present invention, the polymorphism of the ALDH2 gene rs671 and the polymorphism of the ADH2 gene rs1229984 may be detected at the same time, and, for example, in cases where both the ALDH2 gene and the ADH2 gene have the mutations, it may be suggested that the tolerance to alcohol is lower.

### <2> Primers of Present Invention

The primers of the present invention are primers to be used in the detection method of the present invention together with the probe described herein.

In some embodiments, the primers of the present invention are primers for detecting at least one polymorphism selected from the group consisting of the polymorphism of the ALDH2 gene rs671 and the polymorphism of the ADH2 gene rs1229984, comprising the oligonucleotides (P4) and (P5) and/or (P6) and (P7) described below:

(P4) an oligonucleotide comprising a nucleotide sequence of 21 to 60 consecutive nucleotides containing nucleotides 89 to 109 in SEQ ID NO:1 or 2;

(P5) an oligonucleotide comprising a nucleotide sequence complementary to a nucleotide of 20 to 60 consecutive nucleotides containing nucleotides 388 to 407 in SEQ ID NO:1 or 2;

(P6) an oligonucleotide comprising a nucleotide sequence of 46 to 60 consecutive nucleotides containing nucleotides 93 to 138 in SEQ ID NO:13; and

(P7) an oligonucleotide comprising a nucleotide sequence complementary to a nucleotide of 25 to 60 consecutive nucleotides containing nucleotides 214 to 238 in SEQ ID NO:13.

The sequence of each of the primers (P4) to (P7) of the present invention does not need to be completely identical to the above-described sequence, and may be different for only 5, 4, 3, 2 or 1 nucleotides. For example, the primers shown in SEQ ID NOs:7, 8, 19 and 20 are used in the present invention.

### <3> Kit of Present Invention

The kit according to some embodiments of the present invention is a kit to be used for the detection method of the present invention. This kit may comprise the probe described herein for detecting a polymorphism. The kit of the present invention may also be used for judging the capacity to metabolize, and the tolerance to, alcohol.

The detection kit of the present invention may further comprise, in addition to the probe, reagents required for nucleic acid amplification in the detection method of the present invention, especially the above-described primers for amplification using a DNA polymerase.

In the detection kit of the present invention, the probe, primers and other reagents may be contained separately, or a mixture of a part of them may be contained. The present invention is described more concretely by way of Examples below. However, these Examples are merely examples and the present invention is not limited to the Examples.

In the present invention, in terms of the individual sequences in the sample nucleic acids, probes for detecting a polymorphism(s) and primers, matters described based on the complementary relationship between these are applied to the respective sequences and also to the sequences complementary thereto unless otherwise specified. When the matters of the present invention are applied to the sequence complementary to each sequence, the sequence recognized by the complementary sequence is read as the sequence complementary to the corresponding sequence described in the present specification throughout the specification according to the common technical knowledge.

### EXAMPLES

### Example 1

### Detection of Template Oligonucleotides for ALDH2 Using Single Probe

Based on the nucleotide sequence comprising the site of the polymorphism of the ALDH2 gene rs671 (SEQ ID NO:1 (wild type)), probes having C at the 3′ end (which correspond to the wild type (SEQ ID NOs:3 and 5) and the mutant type (SEQ ID NO:4)) and a probe having C at the 5′ end (which corresponds to the wild type (SEQ ID NO:6)) shown in Table 1 were designed. In Table 1, the position of each probe is indicated by its nucleotides in the nucleotide sequence shown in SEQ ID NO:1 in the cases of the wild type, and in the nucleotide sequence shown in SEQ ID NO:2 in the cases of the mutant type. “P” at the 3′ end indicates phosphorylation. Labeling with TAMRA was carried out according to a conventional method.

The sequences of the template oligonucleotides used as the subjects of detection (wild-type sense (SEQ ID NO:9), wild-type antisense (SEQ ID NO:11), mutant-type sense (SEQ ID NO:10) and mutant-type antisense (SEQ ID NO:12)) are shown in Table 1. In Table 1, the position of each oligonucleotide is indicated by its nucleotides in the nucleotide sequence shown in SEQ ID NO:2. In Tables, the nucleotides represented by uppercase letters indicate the position of mutation.

TABLE 1

| SEQ ID NO | Probe name | Sequence(5′→3′) | Nucleotides |
|---|---|---|---|
| 3 | 3T-ALDH2*2-wt-R1- | gttttcacttCagtgtatgcc-(TAMRA) | 261-241 |
| | 21 | | |
| 4 | 3T-ALDH2*2-mt-F1- | ggcatacactAaagtgaaaac-(TAMRA) | 241-261 |
| | 21 | | |
| 5 | 3T-ALDH2*2-mt-R3- | ttttcacttTagtgtatgcc-(TAMRA) | 260-241 |
| | 20 | | |
| 6 | 5T-ALDH2*2-wt-R2- | (TAMRA)-cttCagtgtatgcctgcag-P | 254-236 |
| | 19 | | |
| | Template | | |
| | oligonucleotide | | |
| SEQ ID NO | name | Sequence(5′→3′) | Nucleotides |
| 9 | ALDH2*1 F50 | gggcgagtacgggctgcaggcatacactGaagtgaaaactgtgagt | 223-272 |
| | | gtgg | |
| 10 | ALDH2*2 F50 | gggcgagtacgggctgcaggcatacactAaagtgaaaactgtgagt | 223-272 |
| | | gtgg | |
| 11 | ALDH2*1 R50 | ccacactcacagttttcacttCagtgtatgcctgcagcccgtactcgcc | 272-223 |
| | | c | |
| 12 | ALDH2*2 R50 | ccacactcacagttttcacttTagtgtatgcctgcagcccgtactcgcc | 272-223 |
| | | c | |

Tm analysis was carried out using Smart Cycler (manufactured by Cephied). The composition of the probe solution was as follows. As samples, the following combinations of template oligonucleotides were used. The conditions of the Tm analysis were: 95° C. for 1 second→47° C. for 60 seconds→(47° C.→94° C., 1° C./second).

The excitation wavelength and the detection wavelength in the Tm analysis were 520 to 555 nm and 585 to 700 nm (TAMRA), respectively.

TABLE 2

| Formulation | Total | |
|---|---|---|
| 1 × GeneTaq Buffer | 25 | μl |
| Probe | 0.2 | μM |
| Template oligonucleotide (WT mt)* | 0.4 | μM |

| | Template | Template |
|---|---|---|
| Probe | oligonucleotide (WT) | oligonucleotide (mt) |
| 3T-ALDH2*2-wt-R1-21 | ALDH2*1 F50 | ALDH2*2 F50 |
| (SEQ ID NO: 3) | (SEQ ID NO: 9) | (SEQ ID NO: 10) |
| 3T-ALDH2*2-mt-F1-21 | ALDH2*1 R50 | ALDH2*2 R50 |
| (SEQ ID NO: 4) | (SEQ ID NO: 11) | (SEQ ID NO: 12) |
| 3T-ALDH2*2-mt-R3-20 | ALDH2*1 F50 | ALDH2*2 F50 |
| (SEQ ID NO: 5) | (SEQ ID NO: 9) | (SEQ ID NO: 10) |
| 5T-ALDH2*2-wt-R2-19 | ALDH2*1 F50 | ALDH2*2 F50 |
| (SEQ ID NO: 6) | (SEQ ID NO: 9) | (SEQ ID NO: 10) |

*The template oligonucleotide was prepared by mixing equal amounts of the following WT and mt before use.
*Combinations of the probe and the template oligonucleotide

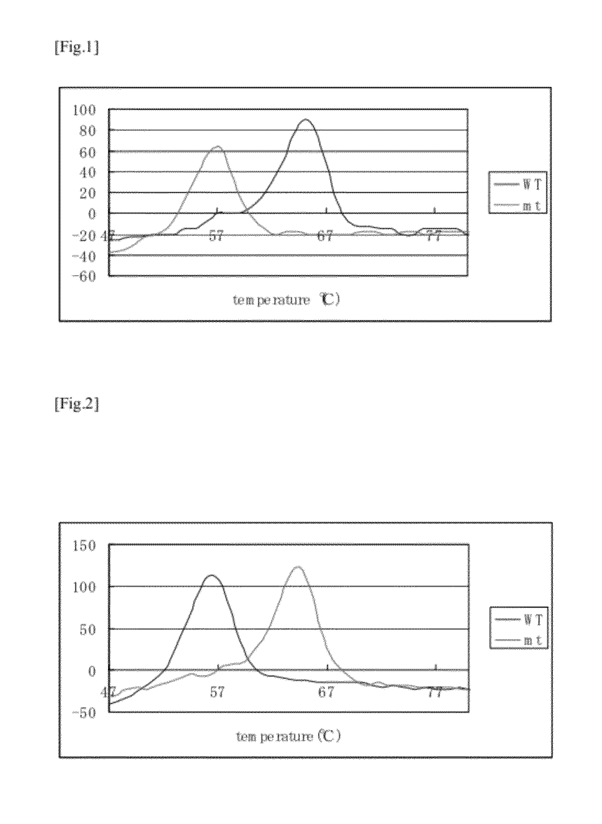

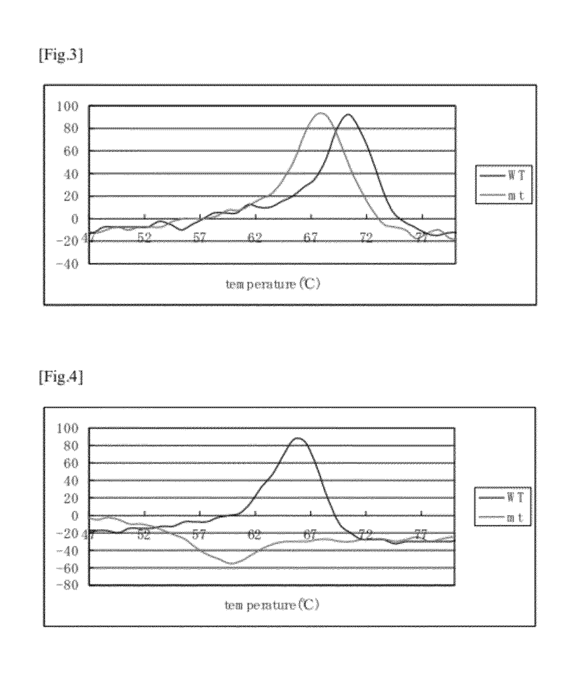

As a result of Tm analysis using the probes shown in Table 1, two clear peaks, that is, the peak of TAMRA corresponding to the wild type and the peak of TAMRA corresponding to the mutant type were observed with 3T-ALDH2*2-wt-R1-21 (SEQ ID NO:3), 3T-ALDH2*2-mt-F1-21 (SEQ ID NO:4) and 3T-ALDH2*2-mt-R3-20 (SEQ ID NO:5) (FIGS. 1 to 3), but the peak of TAMRA corresponding to the mutant type was not observed with 5T-ALDH2*2-wt-R2-19 (SEQ ID NO:6) (FIG. 4).

Therefore, it may be understood that, even in cases where the probe is labeled at C located at its 5′ or 3′ end, the probe sequence may not be arbitrary, and that it is important for the probe to be fluorescently labeled at C located at position 241 or 261, as in the case of the probes having the sequences shown in SEQ ID NOs:3 to 5.

### Example 2

### Detection of Template Oligonucleotides for ADH2 Using Single Probe

Based on the nucleotide sequence comprising the site of the polymorphism of the ADH2 gene rs1229984 (SEQ ID NO:13 (wild type)), probes having C at an end (which correspond to the wild type (SEQ ID NO:16) and the mutant type (SEQ ID NOs:17 and 18)) shown in Table 3 were designed. In Table 3, the position of each probe is indicated by its nucleotides in the nucleotide sequence shown in SEQ ID NO:13 in the cases of the wild type, and in the nucleotide sequence shown in SEQ ID NO:14 in the cases of the mutant type. Labeling with BODIPY FL was carried out according to a conventional method.

Further, the sequences of the template oligonucleotides used as the subjects of detection (which correspond to the wild-type (SEQ ID NOs:21 and 23) and the mutant type (SEQ ID NOs:22 and 24)) are shown in Table 3. In Table 3, the position of each oligonucleotide is indicated by its nucleotides in the nucleotide sequence shown in SEQ ID NO:13 or 14.

In SEQ ID NOs:13 and 14, w represents a or t, and k represents g or t.

TABLE 3

| SEQ ID NO | Probe name | Sequence(5′→3′) | Nucleotides |
|---|---|---|---|
| 16 | 3FL-ADH2-WT- | tctgtcGcacggatgacc-(BODIPY-FL) | 195-212 |
| | F3 | | |
| 17 | 3FL-ADH2-mt- | tctgtcAcacagatgacc-(BODIPY-FL) | 195-212 |
| | F1 | | |
| 18 | 3FL-ADH2-mt- | tgtcAcacagatgaccac-(BODIPY-FL) | 197-214 |
| | F2 | | |
| | Template | | |
| | oligonucleotide | | |
| SEQ ID NO | name | Sequence(5′→3′) | Nucleotides |
| 21 | ADH2*1-F50 | ggtggctgtaggaatctgtcGcacagatgaccacgtggttagtggcaacc | 181-230 |
| 22 | ADH2*2-F50 | ggtggctgtaggaatctgtcAcacagatgaccacgtggttagtggcaacc | 181-230 |
| | ADH2*1-R50 | ggttgccactaaccacgtggtcatctgtgCgacagattcctacagccacc | 230-181 |
| 24 | ADH2*2-R50 | ggttgccactaaccacgtggtcatctgtgTgacagattcctacagccacc | 230-181 |

Tm analysis was carried out using a fully automatic SNPs testing device (trade name: i-densy IS-5310, manufactured by ARKRAY, Inc.). The composition of the PCR reaction solution was as follows. As samples, the following combinations of template oligonucleotides were used. The conditions of the Tm analysis were: 95° C. for 1 second→40° C. for 60 seconds→(40° C.→66° C., 3° C./second).

The excitation wavelength and the detection wavelength in the Tm analysis were 420 to 485 nm and 520 to 555 nm (BODIPY FL), respectively.

TABLE 4

| Formulation) | | Total 25 μl |
|---|---|---|
| 1 × GeneTaq Buffer Probe** | | 0.2 μM |
| Template oligonucleotide(WT · mt)* | | 0.4 μM |
| Probe | Template (WT) | Template (mt) |
| 3FL-ADH2-WT-F3 | ADH2*1-R50 | ADH2*2-R50 |
| (SEQ ID NO: 16) | (SEQ ID NO: 23) | (SEQ ID NO: 24) |
| 3FL-ADH2-mt-F1 | ADH2*1-R50 | ADH2*2-R50 |
| (SEQ ID NO: 17) | (SEQ ID NO: 23) | (SEQ ID NO: 24) |
| 3FL-ADH2-mt-F2 | ADH2*1-R50 | ADH2*2-R50 |
| (SEQ ID NO: 18) | (SEQ ID NO: 21) | (SEQ ID NO: 22) |

*The template oligonucleotide was prepared by mixing equal amounts of the following WT and mt before use.
**Combinations of the probe and the template oligonucleotide

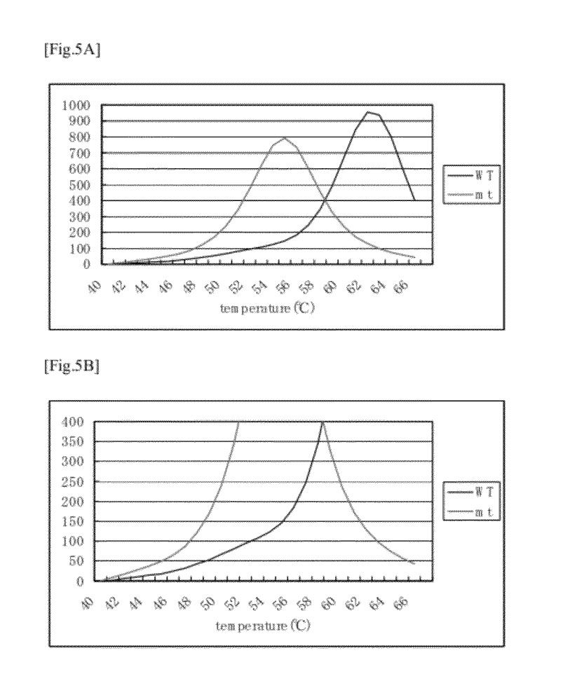

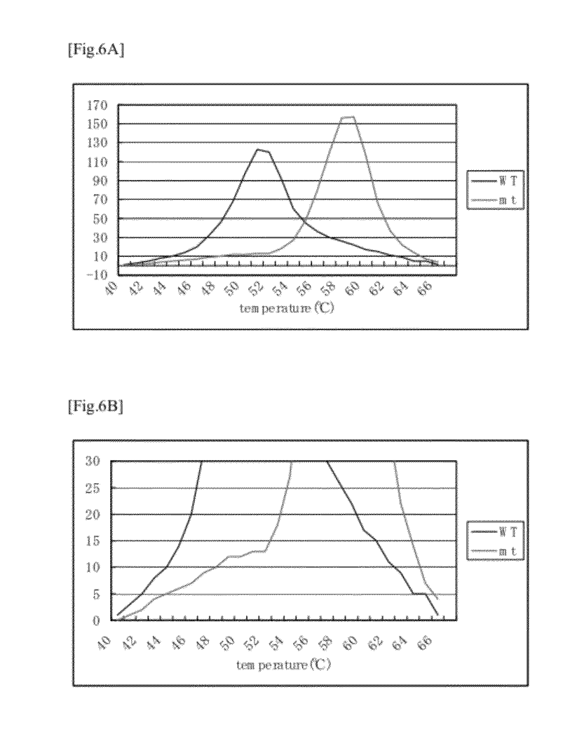

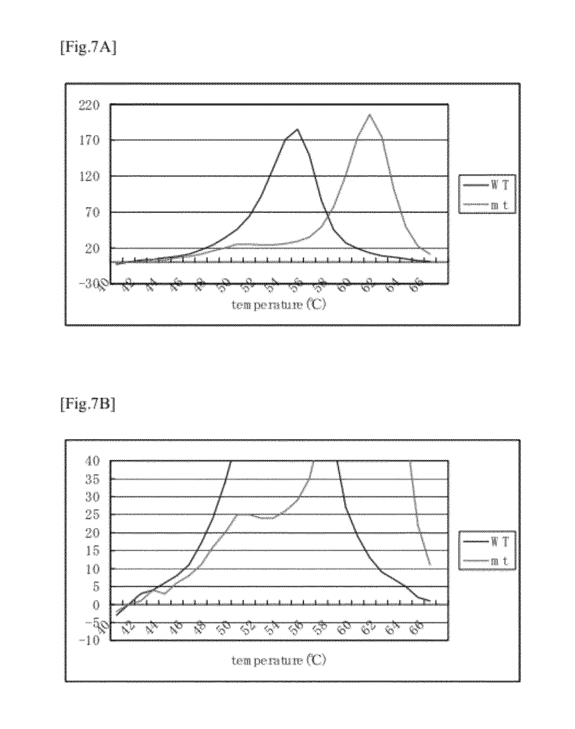

As a result of Tm analysis using the probes shown in Table 3, two clear peaks, that is, the peak of BODIPY FL corresponding to the wild type and the peak of BODIPY FL corresponding to the mutant type were observed with 3FL-ADH2-WT-F3 (SEQ ID NO:16) (FIG. 5), but the peak of BODIPY FL corresponding to the mutant type was not observed with 3FL-ADH2-mt-F1 (SEQ ID NO:17) and 3FL-ADH2-mt-F2 (SEQ ID NO:18) (FIGS. 6A and 7A).

In FIG. 6B, the mt graph shows a noise at 50° C. and a peak at 59° C., and, in such a case, the genotype may be erroneously determined to be heterozygous. Further, in FIG. 7B, the mt graph shows a noise at 51° C. and a peak at 61° C., and, in such a case, the genotype may be erroneously determined to be heterozygous.

Therefore, it may be understood that, even in cases where the probe is labeled at C located at its 3′ end, the probe sequence may not be arbitrary, and that it is important for the probe to be fluorescently labeled at C located at position 212 and to have a wild-type sequence, as in the case of the probe having the sequence shown in SEQ ID NO:16.

### Example 3

### Detection of Oral Swab, Whole Blood or Purified DNA Using Plurality of Probes

As described below, the following primers were used to amplify the polymorphic region from oral swab, whole blood or purified DNA by PCR, and Tm analysis was carried out using the probes having the sequences shown in SEQ ID NOs:4 and 16.

First, based on the nucleotide sequence comprising the site of the polymorphism of the ALDH2 gene rs671 (SEQ ID NO:1 (wild type)), the primers shown in Table 5 were designed such that the polymorphic site may be amplified. In Table 5, the position of each primer is indicated by its nucleotides in the nucleotide sequence shown in SEQ ID NO:1.

Further, based on the nucleotide sequence comprising the site of the polymorphism of the ADH2 gene rs1229984 (SEQ ID NO:13 (wild type)), the primers shown in Table 5 were designed such that the polymorphic site may be amplified. In Table 5, the position of each primer is indicated by its nucleotides in the nucleotide sequence shown in SEQ ID NO:13.

Subsequently, PCR and Tm analysis were carried out using a fully automatic SNPs testing device (trade name: i-densy IS-5310, manufactured by ARKRAY, Inc.). The composition of the PCR reaction solution was as follows. As a sample, the following oral swab, whole blood or purified DNA was used. The conditions of the PCR and Tm analysis were: 95° C. for 60 seconds→(95° C. for 1 second→60° C. for 30 seconds)×50 cycles→95° C. for 1 second→40° C. for 60 seconds→(40° C.→75° C., 1° C./3 seconds).

The excitation wavelength and the detection wavelength in the Tm analysis were 420 to 485 nm and 520 to 555 nm (BODIPY FL), respectively, or 520 to 555 nm and 585 to 700 nm (TAMRA).

TABLE 5

| SEQ ID NO | Primer name | Sequence(5′→3′) | Nucleotides |
|---|---|---|---|
| SEQ ID NO: 7 | ALDH2 F4 | Ctgggagtgtaacccataacc | 89-109 |
| SEQ ID NO: 8 | ALDH2 R9 900-881 | Cagcaggccctgagtccccg | 407-388 |
| SEQ ID NO: 19 | ADH2-F3 | gaaacacaatttcaggaatttgggtatgttaaattcatctagttac | 93-138 |
| SEQ ID NO: 20 | ADH2-R4 | Ggwcaccagkttgccactaaccacg | 238-214 |

| (Reaction solution volume: 50 μl) | |
|---|---|
| 1 × PCR buffer | |
| dNTP | 0.2 mM |
| MgCl₂ | 1.5 mM |
| Taq polymerase (manufactured by ARKRAY, Inc.) | 0.0376U |
| 100 μM ALDH2 F4 | 0.5 μM |
| 100 μM ALDH2 R9 | 1 μM |
| 100 μM ADH2 F3 | 0.5 μM |
| 100 μM ADH2 R4 | l μM |
| 3T-ALDH2*2-mt-F1-21(SEQ ID NO: 4) | 0.2 μM |
| 3FL-ADH2-WT-F3(SEQ ID NO: 16) | 0.1 μM |
| Sample | 4 μl |

*In SEQ ID NO: 20, w represents a or t, and k represents g or t.

### <Preparation of Oral Swab>

In 500 μl of an oral swab-suspending solution (1), oral swab collected by 20 times of rubbing over the both cheeks was suspended. To 90 μl of a diluent (2), 30 μl of the resulting suspension was added, and the resulting mixture was mixed well. A 10-μl aliquot of the mixture was then heated at 95° C. for 5 minutes, to obtain 4 μl of a pretreated oral swab liquid. The resulting liquid was added to the PCR reaction solution, and DNA derived from the pretreated oral swab liquid was used as a template.

TABLE 6

| | ×1 | |
|---|---|---|
| Oral swab-suspending solution (1) | | |
| Tris-HCl (pH 8.0) | 10 | mM |
| EDTA (pH 8.0) | 0.1 | mM |
| SDS | 0.30% | |
| NaN₃ | 0.05% | |
| ProteinaseK | 0.25 | μg/μl |
| Diluent (2) | | |
| Tris-HCl (pH 8.0) | 10 | mM |
| EDTA (pH 8.0) | 0.1 | mM |

### <Preparation of Whole Blood>

In 90 μl of a diluent (1), 10 μl of whole blood was added, and the resulting mixture was mixed well, followed by adding 10 μl of this mixture to 90 μl of a diluent (2). A 17-μl aliquot of the resulting mixture was then heated at 95° C. for 10 minutes, to obtain 4 μl of pretreated whole blood. This was added to the PCR reaction solution, and DNA derived from the pretreated whole blood was used as a template.

TABLE 7

| Diluent(1) | | |
|---|---|---|
| Tris-HCl (pH 8.0) | 10 | mM |
| EDTA (pH 8.0) | 0.1 | mM |
| SDS | 0.30% | |
| Diluent(2) | | |
| Tris-HCl (pH 8.0) | 10 | mM |
| 500 mM EDTA (pH 8.0) | 0.1 | mM |

### <Purified DNA>

In each test, 4 μl of purified DNA at a concentration of 25 copies/μl was added to the PCR reaction solution, as a template.

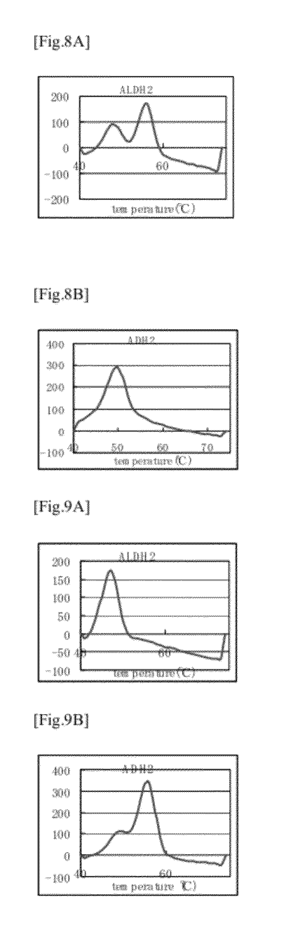

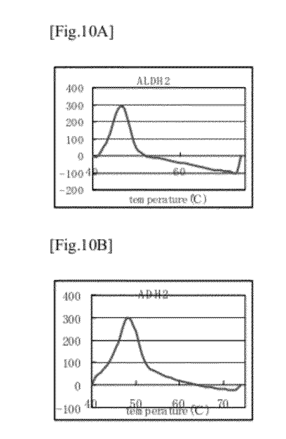

As a result of evaluation of the ALDH2 gene based on fluorescence of TAMRA, the heterozygous oral swab showed the peak of TAMRA corresponding to the wild type and the peak of TAMRA corresponding to the mutant type (FIG. 8A), while the homozygous whole blood and DNA showed only the peak of TAMRA corresponding to the mutant type (FIGS. 9A and 10A).

Further, as a result of evaluation of the ADH2 gene based on fluorescence of BODIPY FL, the wild-type oral swab and DNA showed only the peak of BODIPY FL corresponding to the wild type (FIGS. 8B and 10B), and the heterozygous whole blood showed the peak of BODIPY FL corresponding to the wild type and the peak of BODIPY FL corresponding to the mutant type (FIG. 9B).

Based on the results shown in FIGS. 8 to 10, changes in the fluorescence intensity which may be analyzed by Tm analysis were observed for the polymorphism of the ALDH2 gene and the polymorphism of the ADH2 gene when the probe having the sequence shown in SEQ ID NO:4 in Table 1 and the probe having the sequence shown in SEQ ID NO:16 in Table 3 were used.

That is, in the cases of the polymorphism of the ALDH2 gene, the oral swab with G/A, which is the heterozygote, showed 2 peaks (48° C. and 56° C.), while the whole blood and DNA with A/A, which is a homozygote, showed only 1 peak (48° C.), so that a unique change in the pattern of the amount of change in the fluorescence intensity exists.

Further, also in the cases of the polymorphism of the ADH2 gene, a change in the fluorescence intensity which may be analyzed by Tm analysis was observed. That is, the whole blood with G/A, which is the heterozygote, showed 2 peaks (50° C. and 56° C.), while the oral swab and DNA with G/G, which is the wild-type homozygote, showed only 1 peak (50° C.), so that a unique change in the pattern of the amount of change in the fluorescence intensity exists.

Therefore, by using the probes having the sequences shown in SEQ ID Nos:4 and 16 at the same time, the polymorphism of the ALDH2 gene and the polymorphism of the ADH2 gene may be detected at the same time.

Further, since the probes having the sequences shown in SEQ ID NO:3 and 5 are fluorescently labeled at C located at the 3′ end as in the case of the probe having the sequence of SEQ ID NO:4, and the effect of those probes were demonstrated in Example 1, the polymorphism of the ALDH2 gene and the polymorphism of the ADH2 gene may be detected at the same time also by using the probe having the sequence shown in SEQ ID NO:3 or 5 and the probe having the sequence shown in SEQ ID NO:16 at the same time.

By using the probe described herein, the capacity of a subject to biologically degrade alcohol and the tolerance of the subject to alcohol may be predicted.

1. A labeled probe comprising at least one oligonucleotide selected from the group consisting of oligonucleotides (P1), (P2), and (P3):
(P1) an oligonucleotide comprising a sequence at least about 85% identical to a complementary nucleotide sequence of 11 to 50 nucleotides to nucleotides 241 to 251 of SEQ ID NO:1 or 2;
(P2) an oligonucleotide comprising a sequence at least about 85% identical to a complementary nucleotide sequence of 11 to 50 nucleotides to nucleotides 251 to 261 of SEQ ID NO:1 or 2; and
(P3) an oligonucleotide comprising a sequence at least about 85% identical to a complementary nucleotide sequence of 12 to 50 nucleotides to nucleotides 201 to 212 of SEQ ID NO:13.

2. The probe according to claim 1, wherein
the oligonucleotide (P1) comprises a complementary nucleotide sequence of 11 to 50 nucleotides to nucleotides 241 to 251 of SEQ ID NO:1 or 2;
the oligonucleotide (P2) comprises a complementary nucleotide sequence of 11 to 50 nucleotides to nucleotides 251 to 261 of SEQ ID NO:1 or 2; and
the oligonucleotide (P3) comprises a complementary nucleotide sequence of 12 to 50 nucleotides to nucleotides 201 to 212 of SEQ ID NO:13.

3. The probe according to claim 1, wherein the probe is labeled with a fluorescent dye.

4. The probe according to claim 1, wherein
in said oligonucleotide (P1), the nucleotide corresponding to the nucleotide at position 241 is cytosine labeled with a fluorescent dye; in said oligonucleotide (P2), the nucleotide corresponding to the nucleotide at position 261 is cytosine labeled with a fluorescent dye; and
in said oligonucleotide (P3), the nucleotide corresponding to the nucleotide at position 212 is cytosine labeled with a fluorescent dye.

5. The probe according to claim 1, wherein
said oligonucleotides (P1) has the nucleotide corresponding to the nucleotide at position 241 labeled with a fluorescent dye at the first, second or third position from the 3′ end;
said oligonucleotides (P2) has the nucleotide corresponding to the nucleotide at position 261 labeled with a fluorescent dye at the first, second or third position from the 3′ end; and
said oligonucleotides (P3) has the nucleotide corresponding to the nucleotide at position 212 labeled with a fluorescent dye at the first, second or third position from the 3′ end.

6. The probe according to claim 1, wherein
said oligonucleotides (P1) has the nucleotide corresponding to the nucleotide at position 241 labeled with a fluorescent dye at the 3′ end;
said oligonucleotides (P2) has the nucleotide corresponding to the nucleotide at position 261 labeled with a fluorescent dye at the 3′ end; and
said oligonucleotides (P3) has the nucleotide corresponding to the nucleotide at position 212 labeled with a fluorescent dye at the 3′ end.

7. The probe for detecting a polymorphism according to claim 2, wherein said oligonucleotide emits fluorescence when the oligonucleotide is not hybridized with a target sequence, and the fluorescence intensity decreases or increases when the oligonucleotide is hybridized with the target sequence.

8. The probe for detecting a polymorphism according to claim 6, wherein the fluorescence intensity decreases when said oligonucleotide is hybridized with the target sequence.

9. The probe for detecting a polymorphism according to claim 1, wherein said oligonucleotides (P1), (P2) and (P3) have 12 to 30 nucleotides.

10. The probe for detecting a polymorphism according to claim 1, wherein said oligonucleotides (P1), (P2) and (P3) have 15 to 30 nucleotides.

11. The probe for detecting a polymorphism according to claim 1, wherein said oligonucleotides (P1), (P2) and (P3) have 18 to 30 nucleotides.

12. The probe for detecting a polymorphism according to claim 1, wherein said probe is a probe for melting curve analysis.

13. A method for detecting at least one polymorphism selected from the group consisting of the polymorphism of acetaldehyde dehydrogenase 2 (ALDH2) gene rs671 and the polymorphism of the alcohol dehydrogenase 2 (ADH2) gene rs1229984, comprising
(I) bringing the probe for detection of a polymorphism(s) according to claim 1 into contact with single-stranded nucleic acid in a sample, to allow hybridization of said fluorescently labeled oligonucleotide(s) with said single-stranded nucleic acid, thereby obtaining a hybrid-forming body/bodies;
(II) changing the temperature of the sample containing the hybrid-forming body/bodies to dissociate the hybrid-forming body/bodies, and measuring fluctuation of a fluorescence signal(s) due to the dissociation of the hybrid-forming body/bodies;
(III) determining the Tm value(s), which is/are the dissociation temperature(s) of the hybrid-forming body/bodies, based on the fluctuation of said signal(s); and
(IV) determining based on said Tm value(s) the presence of at least one polymorphism, or the abundance ratio(s) of a nucleic acid(s) having a polymorphism(s), which polymorphism(s) is/are at least one polymorphism selected from the group consisting of the polymorphism of the ALDH2 gene rs671 and the polymorphism of the ADH2 gene rs1229984.

14. The method for detecting a polymorphism(s) according to claim 13, comprising amplifying nucleic acid before said Step (I) or at the same time with said Step (I)

15. A method comprising detecting at least one polymorphism selected from the group consisting of the polymorphism of the ALDH2 gene rs671 and the polymorphism of the ADH2 gene rs1229984, by the method for detecting a polymorphism(s) according to claim 13; and evaluating capacity to metabolize, and/or judging tolerance to, alcohol based on the presence/absence of said polymorphism(s).

16. A kit comprising the probe for detection of a polymorphism(s) according to claim 1, which kit is used for detecting at least one polymorphism selected from the group consisting of genetic polymorphisms of the ALDH2 gene and genetic polymorphisms of the ADH2 gene.

17. The kit for detection of a polymorphism(s) according to claim 14, further comprising a primer that enables amplification using as a template a region in the nucleotide sequence shown in SEQ ID NO:1 or 2 comprising a sequence with which said oligonucleotide (P1), or (P2) hybridizes, and a primer that enables amplification using as a template a region in the nucleotide sequence shown in SEQ ID NO:13 comprising a sequence with which said oligonucleotide (P3) hybridizes.

18. A method using the probe according to claim 1, and primers which are used for detecting at least one polymorphism selected from the group consisting of the polymorphism of the ALDH2 gene rs671 and the polymorphism of the ADH2 gene rs1229984, said primers comprising the oligonucleotides (P4) and (P5) and/or (P6) and (P7) described below:
(P4) an oligonucleotide comprising a nucleotide sequence of 21 to 60 consecutive nucleotides containing nucleotides 89 to 109 in SEQ ID NO:1 or 2;
(P5) an oligonucleotide comprising a nucleotide sequence complementary to a nucleotide of 20 to 60 consecutive nucleotides containing nucleotides 388 to 407 in SEQ ID NO:1 or 2;
(P6) an oligonucleotide comprising a nucleotide sequence of 46 to 60 consecutive nucleotides containing nucleotides 93 to 138 in SEQ ID NO:13; and
(P7) an oligonucleotide comprising a nucleotide sequence complementary to a nucleotide of 25 to 60 consecutive nucleotides containing nucleotides 214 to 238 in SEQ ID NO:13.

19. A method for evaluating capacity to metabolize, and/or judging tolerance to, alcohol, comprising evaluating capacity to metabolize, and/or judging tolerance to alcohol based on the presence/absence of a polymorphism(s), said method comprising detecting at least one polymorphism selected from the group consisting of the polymorphism of the ALDH2 gene rs671 and the polymorphism of the ADH2 gene rs1229984 by the method according to claim 16, and evaluating capacity to metabolize, and/or judging tolerance to, alcohol based on the presence/absence of the polymorphism(s).

20. A reagent kit for detection of at least one polymorphism selected from the group consisting of the polymorphism of the ALDH2 gene rs671 and the polymorphism of the ADH2 gene rs1229984, comprising the probe according to claim 1 and primers comprising the oligonucleotides (P4) and (P5) and/or (P6) and (P7) according to claim 18.
